(19) United States
(12) Patent Application Publication
(10) Pub. No.: US 2019/0355460 A1
(43) Pub. Date: Nov. 21, 2019

(54) System and Method of Providing a Secure Prescription Management

(71) Applicant: SYB International Inc., Toronto (CA)

(72) Inventor: Howard Mann, Toronto (CA)

(21) Appl. No.: 16/379,563

(22) Filed: Apr. 9, 2019

Related U.S. Application Data
(60) Provisional application No. 62/671,932, filed on May 15, 2018.

(51) Int. Cl. G16H 20/10; G16H 10/60; G16H 15/00; G06F 21/62; H04L 9/06

(52) U.S. Cl. CPC G16H 20/10; G16H 10/60; H04L 9/0637; G06F 21/6245; G16H 15/00

(57) ABSTRACT

A system and method of providing a secure prescription management that allows a patient to conveniently purchase a prescription drug. In further detail, the system and method is able to verify if a patient is able to purchase a prescription drug in accordance to his or her medical record and if so, the patient is quickly able to purchase a prescription drug. The system includes a blockchain ledger that is managed by a network of computing nodes. The inclusion of a blockchain ledger provides a blockchain file storage system and a blockchain permission-based ledger which protects medical records and transaction logs of each patient. Moreover, the system and method allows a medical professional to audit the purchase for a prescription drug made by a patient.

The current application claims a priority to the U.S. Provisional Patent application Ser. No. 62/671,932 filed on May 15, 2018.

### FIELD OF THE INVENTION

The present invention relates generally to a data processing. More specifically, the present invention is a method and system to facilitate storage of medical records particular to medical cannabis patients, doctors, and licensed providers.

### BACKGROUND OF THE INVENTION

Electronic medical records are becoming mainstream and are being used by almost all healthcare institutions. As such, the electronic medical records are stored on secure databases to protect the private information related to the patients, including Protected Healthcare Information (PHI). However, regular and manual audits of data stored on traditional databases are required to ensure that PHI is not compromised or has been maliciously altered.

Traditional databases may not be able to compile every addition, change, or deletion in the data into an index history to validate and secure the changes made to the data. Further, audits of medical records and patient records are often long manual processes which may involve several individuals. Further, existing systems and databases may not be automatically updated through connected devices. Further, existing databases may not secure existing data using multiple keys, so as to provide a required level of access to individuals of the confidential patient data.

Therefore, there is a need for improved methods and systems to storage of medical records particular to medical cannabis patients, doctors, and licensed providers that may overcome one or more of the above-mentioned problems and/or limitations. The present invention accomplishes this objective by implementing a blockchain file storage system and a blockchain permission-based ledger. The blockchain file storage system prevents medical records from being accessed or modified by an unwanted user. Additionally, the permission-based ledger allows a use to securely access his or her medical records through the present invention. Moreover, the present invention allows a medical professional to audit financial transactions that reference medical records through the present invention.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

According to some embodiments, a method to facilitate storage of medical records particular to medical cannabis patients, doctors, and licensed providers is disclosed.

Accordingly, the method may include a step of receiving, using a communication device, one or more patient identifiers from a user device for the purpose of registration. In an embodiment, the patients may include patients who may have been prescribed one or more medicines including controlled substances, such as opioids, cannabis, antidepressants, and so on. Further, the method may include a step of receiving, using the communication device, medical records related to the patient. Further, the method may include a step of storing, using a storage device, medical records related to the patient, using blockchain technology. Further, according to some aspects, a system to facilitate storage of medical records particular to medical cannabis patients, doctors, and licensed providers is disclosed.

Accordingly, the system may include a communication device to receive one or more patient identifiers from a user device for the purpose of registration, and medical records related to the patient. Further, the system may include a storage device to store medical records related to the patient, using blockchain technology.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

FIG. 1 is a block diagram illustrating the system of the present invention.

FIG. 2 is a flowchart illustrating the overall method of the present invention.

FIG. 3 is a flowchart illustrating the subprocess of a medical professional auditing a financial transaction.

FIG. 4 is a flowchart illustrating the subprocess of a medical professional accessing the blockchain ledger.

FIG. 5 is a flowchart illustrating the subprocess of retrieving medical records from at least one external third-party server.

FIG. 6 is a flowchart illustrating the subprocess of a patient accessing the blockchain ledger.

FIG. 7 is a flowchart illustrating the subprocess of an existing smart contract including a privacy key and being used to determine how a financial transaction is managed.

FIG. 8 is a flowchart illustrating the subprocess of a new smart contract generating a privacy key and being used to determine how a financial transaction is managed.

FIG. 9 is a flowchart illustrating the subprocess of providing vendor accounts that a patient can select from after selecting a prescription drug.

FIG. 10 is a flowchart illustrating the subprocess of providing vendor accounts that a patient can select from before selecting a prescription drug.

### DETAIL DESCRIPTIONS OF THE INVENTION

All illustrations of the drawings are for the purpose of describing selected versions of the present invention and are not intended to limit the scope of the present invention.

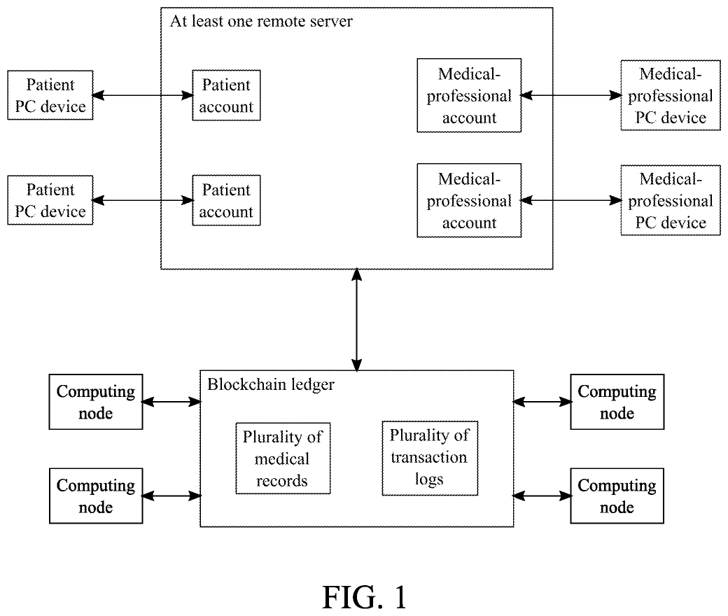

The present invention is a system and method of providing a secure prescription management that allows a patient to conveniently purchase a prescription drug. In further detail, the present invention is able to verify if a patient is able to purchase a prescription drug in accordance to his or her medical record and if so, the patient is quickly able to purchase a prescription drug. With reference to FIG. 1, the system of the present invention is provided with at least one remote server. The remote server is a centralized server that is used to manage the administrative processes of the present invention. The remote server is used to manage a plurality of patient accounts (Step A). Each patient account is associated with a corresponding patient personal computing (PC) device. A patient account allows a user to interact with and use with present invention through the remote server. The patient PC device may be any type of computing device such as, but not limited to, a desktop computer, a mobile device, a smartphone, or an electronic tablet. Additionally, the system of the present invention is provided with a network of computing nodes. The network of computing nodes is a set of computing devices that is used to manage a blockchain ledger (Step B). The network of computing nodes allows for a distributed file storage system which prevents information from being modified. In further detail, the network of computing nodes stores a plurality of medical records and plurality of transaction logs. The plurality of medical records is Protected Healthcare Information (PHI). The plurality of medical records cannot be modified and/or accessed due to being stored on distributed file storage system provided by the network of computing nodes. Each patient account is associated with a corresponding medical record from the plurality of medical records. The plurality of transaction logs is transaction history that includes information such as, but not limited to, a transaction date and financial information. Each patient account is associated with a corresponding transaction log from the plurality of transaction logs. The blockchain ledger is a permission-based distributed ledger that records transaction information.

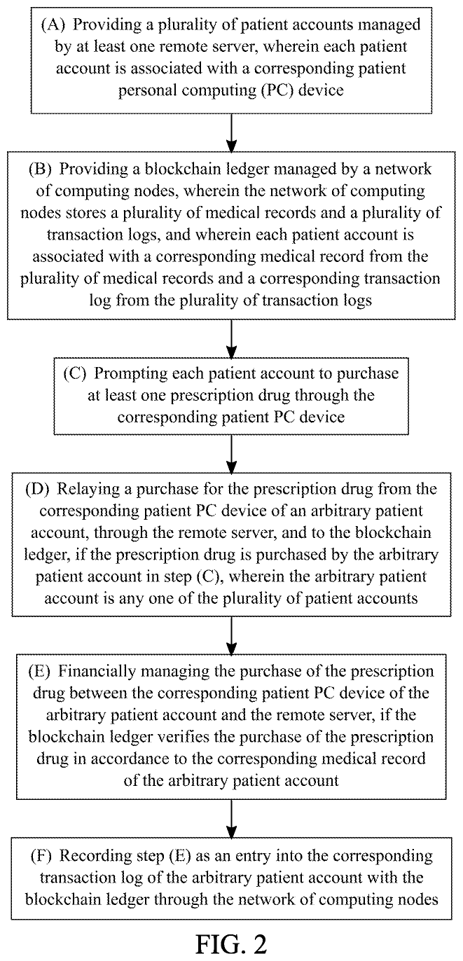

With reference to FIG. 2, the method of the present invention follows an overall process that allows a consumer to easily and conveniently purchase a prescription drug. The corresponding patient PC device prompts each patient account to purchase at least one prescription drug (Step C). The at least one prescription drug may be any type of drug, but preferably is medical marijuana. A purchase of the prescription drug is relayed from the corresponding patient PC device of an arbitrary patient account, through the remote server, and to the blockchain ledger (Step D). This step is processed if the prescription drug is purchased by the arbitrary patient account in Step C. The arbitrary patient account is any one of the plurality of patient accounts. The purchase of the prescription drug is a preparation of the financial transaction for the prescription drug selected by the arbitrary patient account. The purchase of the prescription drug is financially managed between the corresponding patient PC device of the arbitrary patient account and the remote server (Step E). This step occurs if the blockchain ledger verifies the purchase of the prescription drug in accordance to the corresponding medical record of the arbitrary patient account. In further detail, the blockchain ledger references the medical record of the arbitrary patient account to verify if the user of the arbitrary patient account has been given prescription by a medical professional in order to purchase the selected prescription drug. Furthermore, Step E includes a payment process where the user of the arbitrary patient account is able to provide payment information in order to purchase the prescription drug. Step E is recorded as an entry into the corresponding transaction log of the arbitrary patient account with the blockchain ledger through the network of computing nodes (Step F). Step F prevents the financial transaction from Step E from being modified and therefore protects the information of the arbitrary patient account. Additionally, Step F allows the user with the arbitrary account to reference the entry recorded into the corresponding transaction log.

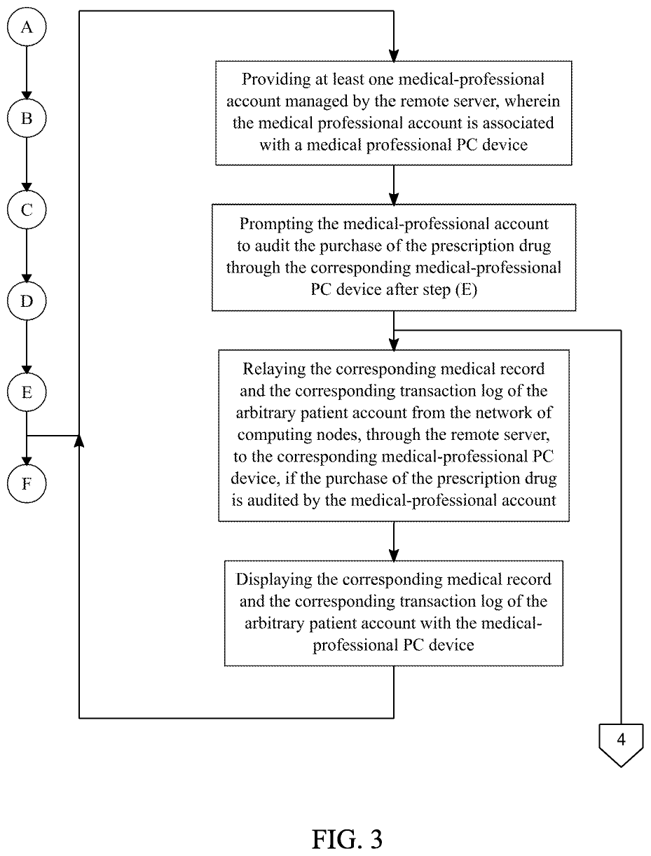

In order for a medical professional to audit the financial transaction of a patient and with reference to FIGS. 1 and 3, at least one medical-professional account is managed by the remote server. The medical-professional account is associated with a medical-professional PC device, which can be, but is not limited to, a desktop computer, a mobile device, a smartphone, or an electronic tablet. The medical-professional account is designated for a user that is, but not limited to, a doctor, a Health Insurance Portability and Accountability (HIPAA) compliant, or other healthcare provider. The medical-professional account is prompted to audit the purchase of the prescription drug through the corresponding medical-professional device after Step E. This step provides the option for the medical-professional account to verify the purchase of the arbitrary patient account. The corresponding medical record and the corresponding transaction log of the arbitrary patient account is relayed from the network of computing nodes, through the remote server, and to corresponding medical-professional PC device. This step is processed if the purchase of the prescription drug is audited by the medical-professional account. In further detail, this step gathers and sends the correct information of the arbitrary patient account in order for the medical-professional account to properly verify the purchase of the arbitrary patient account. The corresponding medical record and the corresponding transaction log of the arbitrary patient account is displayed with the medical-professional PC device. In order for the medical-professional account to reference the correct medical record to verify the purchase of the arbitrary patient account, the corresponding medical record and the corresponding transaction log of the arbitrary patient account are displayed next to each other on the medical-professional PC device.

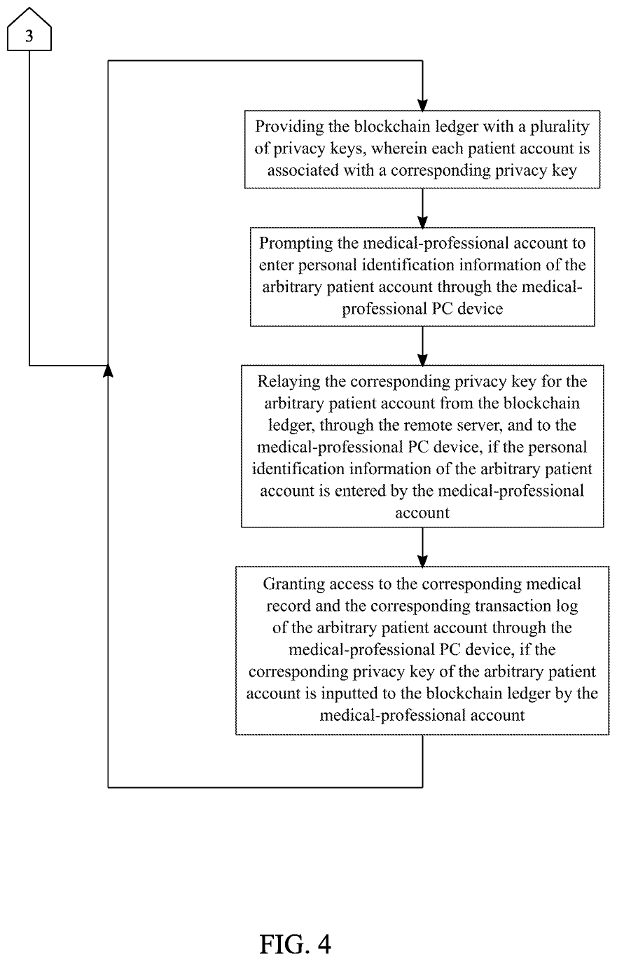

With reference to FIG. 4, the blockchain ledger includes a plurality of privacy keys. Each patient account is associated with a corresponding privacy key. The plurality of privacy keys is a set of access keys that is produced by the blockchain ledger in order to allow the medical-professional account to view the medical record and transaction log of a patient account and in order to allow a patient account to view his or her medical record and/or transaction log. The medical-professional account is prompted to enter personal identification information of the arbitrary patient account through the medical-professional PC device. The personal identification information may be any type of information such as, but not limited to, the name, and/or birthdate of a patient. The corresponding privacy key for the arbitrary patient account is relayed from the blockchain ledger, through the remote server, and to the medical-professional PC device. This step is processed if the personal-identification information of the arbitrary patient account is entered by the medical-professional account. In further detail, this step collects and sends the correct privacy key to medical-professional account. Access is granted to the corresponding medical record and the corresponding transaction log of the arbitrary patient account through the medical-professional PC device. This step is processed if the corresponding privacy key of the arbitrary patient account is inputted to the blockchain ledger by the medical-professional account. In further detail, this step allows the corresponding medical record and the corresponding transaction log of the arbitrary patient account to be securely displayed on the medical-professional device.

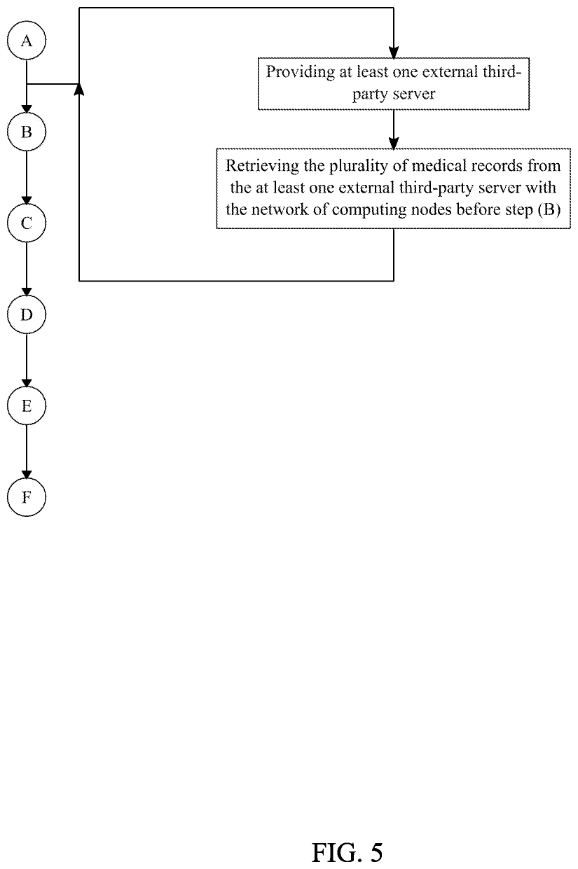

With reference to FIG. 5, at least one external third-party server is provided. The external third-party server may be any server(s) of a medical facility that stores of medical records of the users with patient accounts. The plurality of medical records is retrieved from the at least one external third-party server with the network of computing nodes before Step B. This step is processed in order for the present invention to have access to the medical records of users with patient accounts. Moreover, this allows a user with patient account to view his or her medical record through the present invention and a user with a medical-professional account to view the medical record of his or her patients.

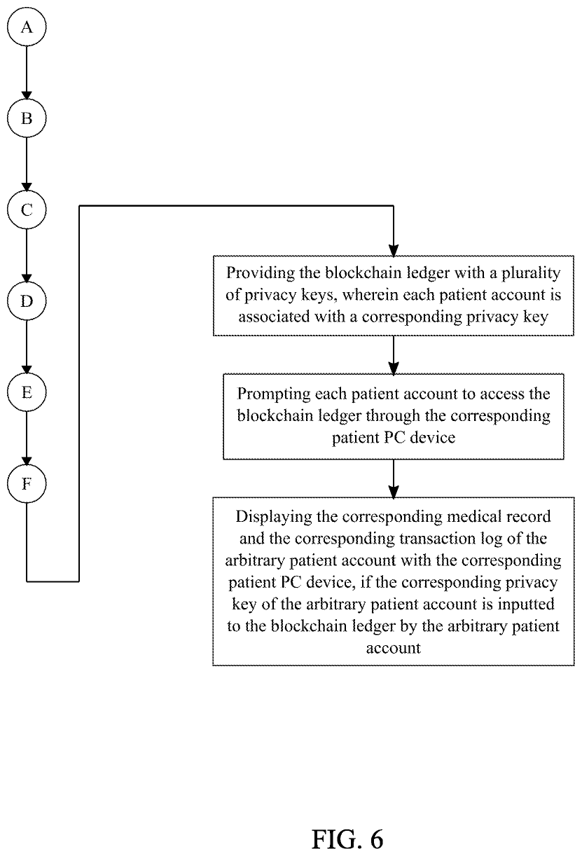

With reference to FIG. 6, each patient account is prompted to access the blockchain ledger through the corresponding patient PC device. This step provides users with a patient account the option to view his or her medical record and/or transaction log through the blockchain ledger. The corresponding medical record and the corresponding transaction log of the arbitrary patient account are displayed with the corresponding patient PC device. This step is processed if the corresponding privacy key of the arbitrary patient account is inputted to the blockchain ledger by the arbitrary patient account. In further detail, this step allows a user with a patient account to view his or her medical record and/or transaction log in order to verify if they have permission to purchase certain prescription drugs and to check their financial transaction history.

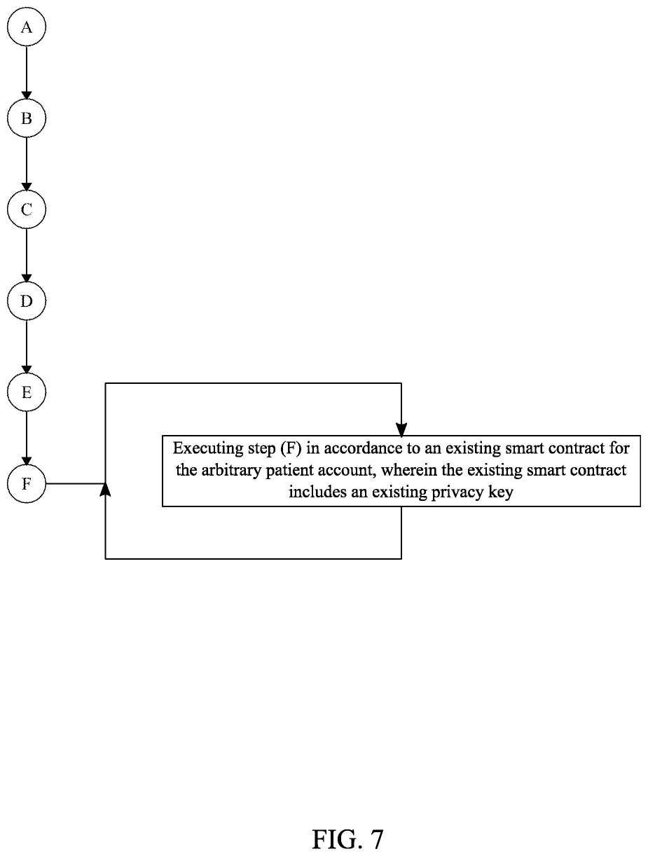

With reference to FIG. 7, Step F is executed in accordance to an existing smart contract for the arbitrary patient account. The existing smart contract includes an existing privacy key. The existing smart contract is used to determine how the financial transactions of a user with a patient account is managed. The existing smart contract is a smart contract which was generated by the blockchain ledger following the first purchase of a user with a patient account. The existing privacy key is an access key that is generated by the smart contract and assigned to the corresponding user with a patient account. The existing privacy key allows the corresponding user with a patient account to access the blockchain ledger.

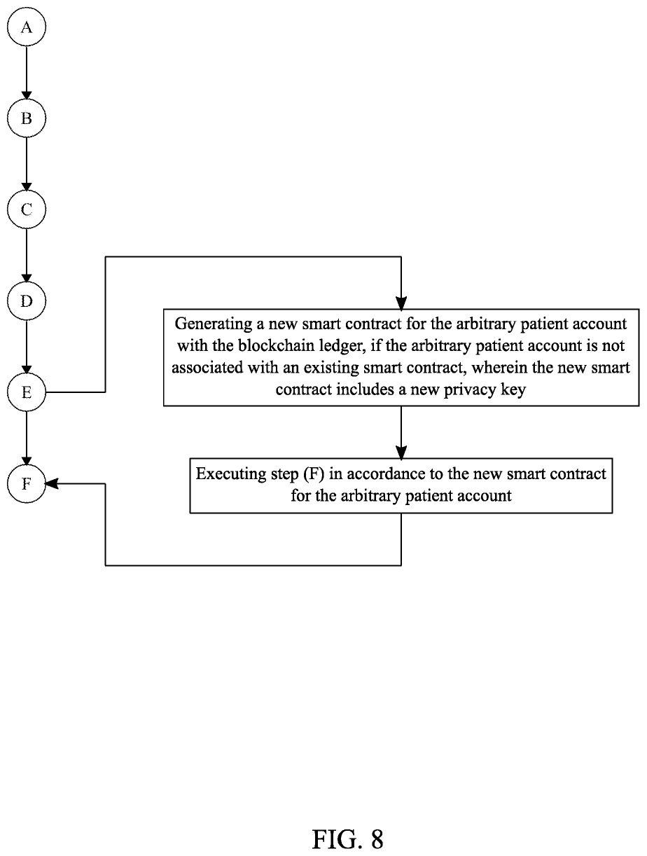

If a user with a patient account has not made a first purchase and with reference to FIG. 8, a new smart contract is generated for the arbitrary patient account with the blockchain ledger. This step is processed if the arbitrary patient account is not associated with an existing smart contract or if the user of the arbitrary patient account is not satisfied with the existing smart contract. The new smart contract includes a new privacy key. The new smart contract is a smart contract that is generated in order to detail how the financial transactions of a user are handled thereinafter. The new privacy key is generated in order to allow a corresponding user with a patient account to view his or her transactions through the blockchain ledger. Step F is consequently executed in accordance to the new smart contract for the arbitrary patient account.

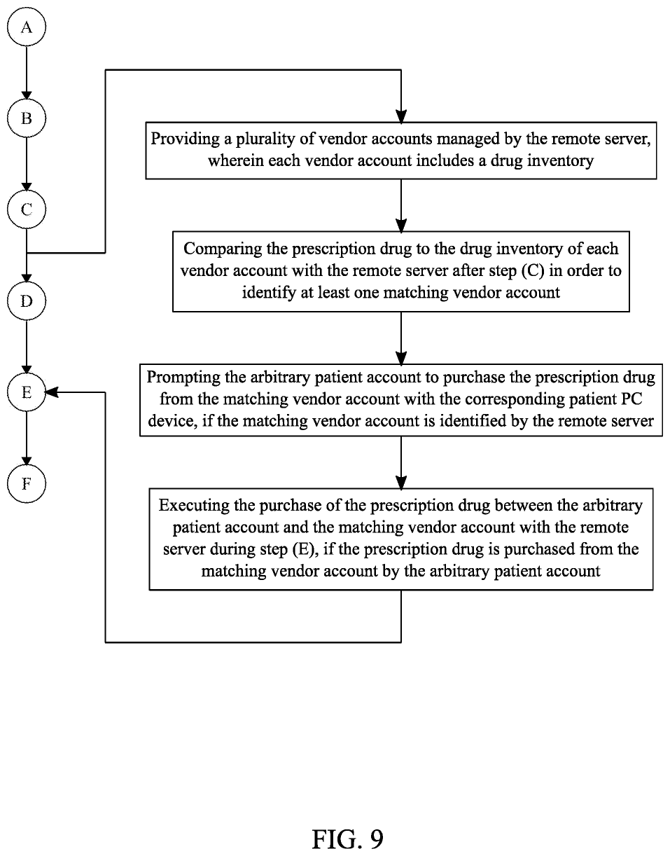

With reference to FIG. 9, the remote server is used to manage a plurality of vendor accounts. Each vendor account includes a drug inventory. The plurality of vendor accounts is a set of user accounts that allows online vendors to interact with the present invention by selling prescription drugs on the platform. The drug inventory is preferably a list of available prescription drugs provided by each vendor account. The prescription drug is compared to the drug inventory of each vendor account with the remote server after Step C in order to identify at least one matching vendor account. The at least one matching vendor account is one or more vendor accounts that sells the prescription drug chosen by the arbitrary patient account. The arbitrary patient account is prompted to purchase the prescription drug from the matching vendor account with the corresponding patient PC device. This step is processed if the matching vendor account is identified by the remote server. In further detail, this step allows the arbitrary patient account to choose the matching vendor account in order to purchase the prescription drug. The purchase of the prescription drug is executed between the arbitrary patient account and the matching vendor account with the remote server during Step E. This step is processed if the prescription drug is purchased from the matching vendor account by the arbitrary patient account.

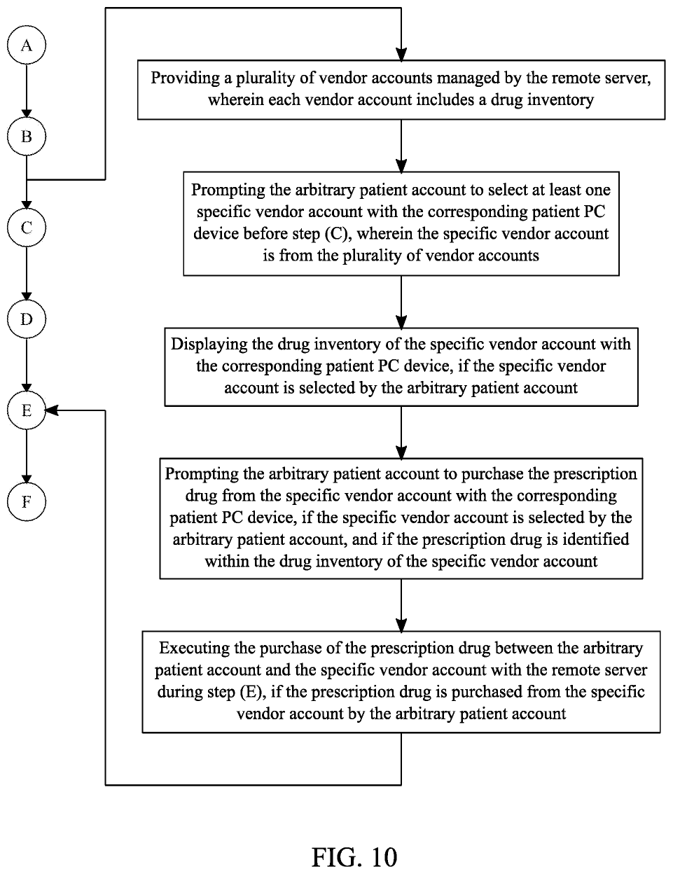

Alternatively and with reference to FIG. 10, the arbitrary patient account is prompted to select at least one specific vendor account with the corresponding patient PC device before Step C. The specific vendor account is from the plurality of vendor accounts. In further detail, the arbitrary patient account is provided a list of vendor accounts and choose at least one specific vendor account from the list. The drug inventory of the specific vendor account is displayed with the corresponding patient PC device. This step is processed if the specific vendor account is selected by the arbitrary patient account. In further detail, the arbitrary patient account is provided a list of prescription drugs that are available to purchase from the specific vendor account. The arbitrary patient account is prompted to purchase the prescription drug from the specific vendor account with the corresponding patient PC device. This step is processed if the specific vendor account is selected by the arbitrary patient account and if the prescription drug is identified within the drug inventory of the specific vendor account. In further detail, the arbitrary patient account is given the option to choose a prescription drug from the drug inventory of the specific vendor account. The purchase of the prescription drug is executed between the arbitrary patient account and the specific vendor account with the remote server during Step E. The step is processed if the prescription drug is purchased from the specific vendor account by the arbitrary patient account.

In another embodiment of the present invention, a vendor account from the plurality of vendor accounts may be associated with a dispensary store location or a dispensary device. In further detail, the present invention is integrated into the financial system of a dispensary store location or a dispensary device. This allows a user with a patient account to quickly and conveniently purchase a prescription drug in person. Furthermore, this removes the multiple steps a person must take in order to purchase a prescription drug.

Although the invention has been explained in relation to its preferred embodiment, it is to be understood that many other possible modifications and variations can be made without departing from the spirit and scope of the invention as hereinafter claimed.

What is claimed is:

1. A method of providing a secure prescription management, the method comprises the steps of:
(A) providing a plurality of patient accounts managed by at least one remote server, wherein each patient account is associated with a corresponding patient personal computing (PC) device;
(B) providing a blockchain ledger managed by a network of computing nodes, wherein the network of computing nodes stores a plurality of medical records and a plurality of transaction logs, and wherein each patient account is associated with a corresponding medical record from the plurality of medical records and a corresponding transaction log from the plurality of transaction logs;
(C) prompting each patient account to purchase at least one prescription drug through the corresponding patient PC device;
(D) relaying a purchase for the prescription drug from the corresponding patient PC device of an arbitrary patient account, through the remote server, and to the blockchain ledger, if the prescription drug is purchased by the arbitrary patient account in step (C), wherein the arbitrary patient account is any one of the plurality of patient accounts;
(E) financially managing the purchase of the prescription drug between the corresponding patient PC device of the arbitrary patient account and the remote server, if the blockchain ledger verifies the purchase of the prescription drug in accordance to the corresponding medical record of the arbitrary patient account; and
(F) recording step (E) as an entry into the corresponding transaction log of the arbitrary patient account with the blockchain ledger through the network of computing nodes.

2. The method of providing a secure prescription management, the method as claimed in claim 1 comprises the steps of:
providing at least one medical-professional account managed by the remote server, wherein the medical-professional account is associated with a medical-professional PC device;
prompting the medical-professional account to audit the purchase of the prescription drug through the corresponding medical-professional PC device after step (E);
relaying the corresponding medical record and the corresponding transaction log of the arbitrary patient account from the network of computing nodes, through the remote server, to the corresponding medical-professional PC device, if the purchase of the prescription drug is audited by the medical-professional account; and
displaying the corresponding medical record and the corresponding transaction log of the arbitrary patient account with the medical-professional PC device.

3. The method of providing a secure prescription management, the method as claimed in claim 2 comprises the steps of:
providing the blockchain ledger with a plurality of privacy keys, wherein each patient account is associated with a corresponding privacy key;
prompting the medical-professional account to enter personal identification information of the arbitrary patient account through the medical-professional PC device;
relaying the corresponding privacy key for the arbitrary patient account from the blockchain ledger, through the remote server, and to the medical-professional PC device, if the personal-identification information of the arbitrary patient account is entered by the medical-professional account; and
granting access to the corresponding medical record and the corresponding transaction log of the arbitrary patient account through the medical-professional PC device, if the corresponding privacy key of the arbitrary patient account is inputted to the blockchain ledger by the medical-professional account.

4. The method of providing a secure prescription management, the method as claimed in claim 1 comprises the steps of:
providing at least one external third-party server; and
retrieving the plurality of medical records from the at least one external third-party server with the network of computing nodes before step (B).

5. The method of providing a secure prescription management, the method as claimed in claim 1 comprises the steps of:
providing the blockchain ledger with a plurality of privacy keys, wherein each patient account is associated with a corresponding privacy key;
prompting each patient account to access the blockchain ledger through the corresponding patient PC device; and
displaying the corresponding medical record and the corresponding transaction log of the arbitrary patient account with the corresponding patient PC device, if the corresponding privacy key of the arbitrary patient account is inputted to the blockchain ledger by the arbitrary patient account.

6. The method of providing a secure prescription management, the method as claimed in claim 1 comprises the steps of:
executing step (F) in accordance to an existing smart contract for the arbitrary patient account, wherein the existing smart contract includes an existing privacy key.

7. The method of providing a secure prescription management, the method as claimed in claim 1 comprises the steps of:
generating a new smart contract for the arbitrary patient account with the blockchain ledger, if the arbitrary patient account is not associated with an existing smart contract, wherein the new smart contract includes a new privacy key; and
executing step (F) in accordance to the new smart contract for the arbitrary patient account.

8. The method of providing a secure prescription management, the method as claimed in claim 1 comprises the steps of:
providing a plurality of vendor accounts managed by the remote server, wherein each vendor account includes a drug inventory;
comparing the prescription drug to the drug inventory of each vendor account with the remote server after step (C) in order to identify at least one matching vendor account;
prompting the arbitrary patient account to purchase the prescription drug from the matching vendor account with the corresponding patient PC device, if the matching vendor account is identified by the remote server; and
executing the purchase of the prescription drug between the arbitrary patient account and the matching vendor account with the remote server during step (E), if the prescription drug is purchased from the matching vendor account by the arbitrary patient account.

9. The method of providing a secure prescription management, the method as claimed in claim 1 comprises the steps of:
providing a plurality of vendor accounts managed by the remote server, wherein each vendor account includes a drug inventory;
prompting the arbitrary patient account to select at least one specific vendor account with the corresponding patient PC device before step (C), wherein the specific vendor account is from the plurality of vendor accounts;
displaying the drug inventory of the specific vendor account with the corresponding patient PC device, if the specific vendor account is selected by the arbitrary patient account;
prompting the arbitrary patient account to purchase the prescription drug from the specific vendor account with the corresponding patient PC device, if the specific vendor account is selected by the arbitrary patient account, and if the prescription drug is identified within the drug inventory of the specific vendor account; and
executing the purchase of the prescription drug between the arbitrary patient account and the specific vendor account with the remote server during step (E), if the prescription drug is purchased from the specific vendor account by the arbitrary patient account.
